(19) United States
(12) Patent Application Publication
(10) Pub. No.: US 2022/0168209 A1
(43) Pub. Date: Jun. 2, 2022

(54) Sunscreen Compositions

(71) Applicant: ELC MANAGEMENT LLC, Melville, NY (US)

(72) Inventors: Riana Marie Kuttler, Lindenhurst, NY (US); Linda Josephine Najdek, East Islip, NY (US); Carmen Castillo Bucci, Melville, NY (US)

(21) Appl. No.: 17/535,556

(22) Filed: Nov. 24, 2021

Related U.S. Application Data
(60) Provisional application No. 63/119,630, filed on Nov. 30, 2020.

(51) Int. Cl. A61K 8/81; A61K 8/73; A61Q 17/04; A61K 8/37; A61K 8/35

(52) U.S. Cl. CPC A61K 8/8152; A61K 8/8182; A61K 8/73; A61Q 17/04; A61K 2800/522; A61K 8/35; A61K 2800/30; A61K 2800/594; A61K 8/37

(57) ABSTRACT

A sunscreen composition comprising one or more sunscreen actives, and at least two film formers is provided. The at least two film formers include an acrylates copolymer and a xanthan gum. The ratio of the acrylates copolymer to the xanthan gum is in a range of about 15:1 to about 1:1. The sunscreen composition provides water-resistant properties and very water-resistant properties after water immersion. The sunscreen composition can be present as a spray, cream, serum, lotion or continuous lotion spray.

### FIELD

The present disclosure relates generally to photoprotective compositions. More particularly, the disclosure relates to water-resistant photoprotective sunscreen compositions.

### BACKGROUND

Sunscreen compositions are applied to the human skin to protect the skin from the sun's ultraviolet (UV) rays that can cause erythema, also known as sunburn. Erythema or sunburn can result in reddening of the skin. Sunlight or ultraviolet radiation in the UV-B range has a wavelength of 290 nm to 320 nm and is known to be the primary cause of sunburn. Ultraviolet rays at a wavelength of 320 nm to 400 nm (UV-A radiation) will cause tanning of the skin and can also damage or harm the skin.

Sunscreen compositions are typically formulated to inhibit skin damage and protect the skin from the sun's UV rays. Sunscreen compositions generally filter or block the harmful UVA and UVB rays that damage the skin. However, water exposure and other adverse conditions such as sweat, can diminish protection as the composition can wash off the skin.

While currently available sunscreens can protect against both UVB and UVA radiation, certain sunscreen actives may be undesirable in cosmetic compositions for various reasons. For example, oxybenzone (i.e., benzophenone-3) has been used as a sunscreen agent to provide broad-spectrum ultraviolet coverage, including protection against UVB and UVA rays (short wave). Despite its photoprotective qualities, much debate surrounds the use of oxybenzone in cosmetic compositions because of its potential hormonal and photoallergenic effects, which has led many countries to regulate its use.

Also, mild sweating or swimming is often sufficient to remove most commercially available sunscreen formulations from a person's skin, necessitating repeated applications to the skin. The need to repeatedly apply sunscreen can be inconvenient, and as such, the consumers may delay or forget reapplication of the sunscreen, resulting in sunburn. Furthermore, to achieve water-resistant claims for sunscreens, it is often necessary to add additional levels of sunscreen active ingredients to the compositions to ensure that the desired Sun Protection Factor (SPF) is maintained even after immersion in water. This can be expensive, aesthetically unpleasant and may present uncomfortable feel on the skin.

Therefore, as described above, currently available sunscreen compositions include sunscreen actives that are photo-allergens or irritants and harmful to the skin. The sunscreen compositions result in delayed reapplication, causing sunburn and often require additional levels of active ingredients or agents to obtain the desired Sun Protection Factor (SPF). Accordingly, there is a need for improved photoprotective and water-resistant sunscreen compositions.

### SUMMARY

A sunscreen composition comprising one or more sunscreen actives, and at least two film formers is provided. The at least two film formers include an acrylates copolymer and a xanthan gum, wherein the ratio of the acrylates copolymer to the xanthan gum is about 15:1 to about 1:1. The sunscreen composition provides very water-resistant properties upon application and after or subsequent to water immersion. The sunscreen composition is free of oxybenzone and octocrylene.

The xanthan gum includes dehydroxanthan gum and the composition includes a polyvinylpyrrolidone (PVP) polymer, wherein the PVP polymer is VP/eicosene copolymer or tricontanyl PVP. The tricontanyl PVP is present in the composition from about 0.075% to about 2.0% (w/w) by the total weight of the sunscreen composition.

The composition further includes a rosinate resin, wherein the rosinate resin is methyl dihydroabietate. The ratio of the PVP polymer to the rosinate resin is from about 15:1 to about 1:15. The composition further includes one or more sunscreen actives, including avobenzone, homosalate, salicylate derivatives, and combinations thereof. The sunscreen actives are present in an amount of about 3% to about 30% (w/w) by the total weight of the composition. The sunscreen composition may be in the form of a spray, lotion, serum, cream or continuous lotion spray. An SPF rating from about 75% to about 100% is retained upon application on the skin and following immersion in water for at least eighty minutes, thereby providing very water-resistant properties to the sunscreen composition.

### DETAILED DESCRIPTION

The dimensions and values disclosed herein are not to be understood as being strictly limited to the exact numerical values recited. Instead, unless otherwise specified, each such dimension is intended to mean both the recited value and a functionally equivalent range surrounding that value. Except in operating and comparative examples, or where otherwise explicitly indicated, all numbers in this description indicating amounts or ratios of material or conditions of reaction, physical properties of materials and/or use are to be understood as modified by the word “about”. All amounts are by weight of the final composition, unless otherwise specified.

As used herein, the term “sunscreen” refers to a composition comprising a compound or an active that has photoprotection efficacy. The sunscreens may be organic or inorganic. As used herein, UVA sunscreens refer to a composition that may comprise one or more UVA sunscreen compounds. The term “UVA sunscreen” refers to a composition or a compound that blocks UV radiation in the wavelength range of about 320 to 400 nm. The term “UVB sunscreen” refers a composition or a compound that blocks UV radiation in the wavelength range of about 290 to 320 nm.

As used herein, the term “continuous spray system” refers to compositions having a flowable consistency capable of being discharged or dispersed from an aerosol, an atomizer or the like.

For sunscreen products claiming to be “water resistant,” the advertised SPF value must be determined following 40-minutes of water immersion, and for products claiming to be “very water resistant”, the advertised SPF rating must be determined following 80-minutes of water immersion, which is also referred to herein as the post-immersion SPF. Typically, in order to produce a sunscreen composition having a desired post-immersion SPF rating, it is necessary to formulate the sunscreen composition with a static (i.e., pre-immersion) SPF that is significantly higher than the desired post-immersion SPF. This is performed with an expectation that anywhere from about 25% to about 50% of the composition's SPF rating will be lost following the 80-minute water immersion. As a result, additional levels of sunscreen actives must be added to achieve the needed high static SPF rating and the desired post-immersion SPF.

Advantageously, the compositions disclosed herein provide for methods of protecting the human skin against UV rays with improved water resistance that meet the very water-resistant criteria according to the COLIPA and the FDA guidelines. Moreover, the compositions are very water resistant (i.e., eighty minutes water-resistant) when free of oxybenzone and octocrylene.

Further, the compositions comprise a combination of film formers, such as acrylates, xanthan gums, polyesters, rosinate resins, and/or polyvinylpyrrolidone (PVP) polymers in a carrier to provide very water resistant (80-minutes) properties to the composition. The film formers may be included in a specific ratio range. Furthermore, the compositions may be sunscreen compositions having physical and/or chemical sunscreen agents, free of octocrylene or oxybenzone, both of which are known to be photo-allergens/irritants. Advantageously, the compositions also have a lower amount of sunscreen active(s) than that are typically needed to achieve the desired SPF value.

Exemplary film formers include, without limiting, acrylates, such as acrylates copolymers; xanthan gums, such as, without limiting, dehydroxanthan gum; polyesters, such as polyester-8 and rosinate resins, such as, without limiting, methyl dihydroabietate, and pine resins.

The compositions may further comprise PVP polymers, such as alkylated PVP polymers, rosinate resins and other film formers. Other film formers include starches, polyurethanes, silicone resins etc. PVP polymers, rosinate resins and xanthan gums are the film formers that may be utilized in the compositions.

Advantageously, the compositions utilizing a specific ratio range of film formers provide the effective water-resistant properties. The ratio of an acrylates copolymer to a xanthan gum may be in the range of about 15:1 to about 1:1. The compositions may further include other film formers such as PVP polymers, and rosinate resins such that ratio of a PVP polymer to a rosinate resin may be in the range of about 15:1 to about 1:15. The PVP polymer can be triacontanyl PVP, VP/eicosene copolymer or others.

In certain compositions disclosed herein, the film formers include, PVP polymers, in addition to an acrylates copolymer and a xanthan gum. If present, PVP polymers such as triacontanyl PVP may be included in the composition at about 0.075% to about 2% (w/w) by the total weight of the composition. In certain compositions, PVP polymers such as VP/eicosene copolymer may be included in the composition at about 0.05% to about 3% (w/w) by the total weight of the composition, about 0.15% to about 3% (w/w) of the total weight of the composition. In certain other compositions disclosed herein, the film formers include, PVP polymers and rosinate resins, in addition to an acrylates copolymer to a xanthan gum. If present, rosinate resins may be included in the composition at about 0.005% to about 4% (w/w) by the total weight of the composition, preferably at about 0.015% to about 3% (w/w) of the total weight of the composition. As described above, the ratio of a PVP polymer to a rosinate resin may be in the range of about 15:1 to about 1:15, including all ranges and sub-ranges within.

Exemplary sunscreens that may be used herein include one or more sunscreen actives, including UV-A sunscreen compounds that block UV radiation in the wavelength range of about 320 nanometer (nm) to 400 nm (e.g., avobenzone), and UVB sunscreen compounds that block UV radiation in the wavelength range of from about 290 nm to 320 nm (e.g., homosalate and salicylate derivatives). Further examples of suitable UVA sunscreens include 4-methyldibenzoylmethane, 2-methyldibenzoylmethane, 4-isopropyldibenzoylmethane, 4-tert-butyldibenzoylmethane, 2,4-dimethyldibenzoylmethane, 2,5-dimethyldibenzoylmethane, 4,4′diisopropylbenzoylmethane, 4-tert-butyl-4′-methoxydibenzoylmethane, 4,4′-diisopropylbenzoylmethane, 2-methyl-5-isopropyl-4′-methoxydibenzoymethane, 2-methyl-5-tert-butyl-4′-methoxydibenzoylmethane, and 4-tert-butyl-4′-methoxydibenzoylmethane, also referred to as Avobenzone. Other suitable UVA sunscreens include dicamphor sulfonic acid derivatives, such as ecamsule, a sunscreen, which is terephthalylidene dicamphor sulfonic acid.

As disclosed herein, the compositions of the present disclosure are free of octocrylene. Other suitable UVB chemical sunscreens include benzylidene camphor derivatives as set forth in U.S. Pat. No. 3,781,417, which is hereby incorporated by reference in its entirety.

Compositions herein may also include organic and inorganic sunscreen actives such as, for example, titanium dioxide and zinc oxide. The organic sunscreen active(s) can include, without limiting, avobenzone, homosalate, salicylate derivatives, and combinations thereof. The sunscreen compositions can include sunscreen actives present in an amount of about 3% to 30% (w/w) by the total weight of the sunscreen composition.

The sunscreen compositions may further comprise a cosmetically acceptable vehicle/carrier. Substances formulated into the sunscreen compositions include, but are not limited to, moisturizing agents, astringent agents, chelating agents, surfactants, emollients, preservatives, stabilizers, humectants, fragrances, etc. Such vehicles aid the formation of a protective layer on the skin. Non-limiting examples of suitable vehicles include emulsions (e.g., oil-in-water, water-in-oil, silicone-in-water, water-in-silicone, water-in-oil-in-water, oil-in-water, oil-in-water-in-oil, oil-in-water-in-silicone, etc.), creams, lotions, solutions, anhydrous bases (e.g., lipsticks and powders), gels, ointments, pastes, milks, liquids, aerosols, and solid forms. The composition may be in an oil-in-water system in the form of a continuous spray system (i.e., non-shear thickening). The vehicle or vehicles can be present in the compositions in an amount ranging from about 70% to about 97%, from about 75% to about 95%, or from 50% to about 80% (w/w) by the total weight of the composition, including all subranges within.

Compositions may also include one or more sunscreen solubilizers and/or stabilizers. Exemplary compounds used to solubilize and stabilize the sunscreen agents may include C12-15 alkyl benzoate, butyloctyl salicylate, ethylhexyl methoxycrylene, PPG-15 stearyl ether benzoate, dicaprylyl carbonate.

Compositions herein may include additional active agents. The type of active agents added can be any that are suitable for use in a topical composition. For example, the composition can further comprise moisturizing actives, agents used to treat age spots, keratoses and wrinkles, as well as anti-acne agents, antibacterial agents, antiyeast agents, antifungal agents, antidandruff agents, antidermatitis agents, antipruritic agents, anti-irritant agents, anti-dry skin agents, antiseborrheic agents, antiaging agents, anti-wrinkle agents, additional sunscreen agents, antihistamine agents, skin lightening agents, depigmenting agents, wound-healing agents, vitamins, or self-tanning agents. The above-described active agents are optional components of the sunscreen compositions and may be omitted without materially affecting the photo-protective function of the sunscreen composition.

The sunscreen compositions can also include emollients which may be, but not limited to, stearyl alcohol, cetyl alcohol, oleyl alcohol, isocetyl alcohol, fatty alcohols, propane-1,2-diol, butane-1,3-diol, octadecan-2-ol, glyceryl monostearate, isopropyl isostearate, stearic acid, isostearic acid, isocetyl stearate, isopropyl stearate, butyl stearate, isopropyl laurate, hexyl laurate, decyl oleate, isobutyl palmitate, cetyl palmitate, isopropyl palmitate, palmitic acid, dimethylpolysiloxane, glyceryl monoricinoleate, di-n-butyl sebacate, isopropyl myristate, butyl myristate, myristyl myristate, isopropyl linoleate, lauryl lactate, myristyl lactate, polyethylene glycol, triethylene glycol, lanoline, acetylated lanolin, sesame oil, coconut oil, arrachis oil, castor oil, mink oil, mineral oil, and petroleum.

Preservatives can also be added to the sunscreen compositions to provide a prolonged shelf life. Suitable preservatives include, but are not limited to, potassium sorbate, imidazolidinyl urea, p-hydroxy benzoate, esters of p-hydroxybenzoic acid, CTFA designation parabens, ethylhexylglycerin, caprylyl glycol/phenoxyethanol/hexylene glycol, etc. Other preservatives suitable for use in the sunscreen compositions are disclosed in the International Cosmetic Ingredient Dictionary and Handbook, twelfth edition, 2004, the entire disclosure of which is herein incorporated by reference.

Humectants which may be used include, but not limited to, polyhydric alcohols including, glycerol, polyalkylene glycols, and alkylene polyols and mixtures thereof, hyaluronic acid, urea, glycerin, sorbitol, sodium 2-pyrrolidone-5-carboxylate, soluble collagen, dibutylphthalate and gelatin.

The sunscreen compositions may optionally comprise a fragrance in an amount sufficient to make the composition more appealing to the consumer. Preferably, the fragrance is present in an amount from about 0.01% to about 10% (w/w) by total weight of the composition.

The sunscreen compositions may also include powders such as organic or inorganic powders, soft focus powders, plate-like non-spherical powders such as bismuth oxychloride, boron nitride, barium sulfate, mica, sericite, muscovite, synthetic mica, titanium oxide coated mica, titanium oxide-coated bismuth oxychloride, titanium oxide coated talc, platelet iron oxides, metal powders such as aluminum, lauroyl lysine and platelet talc. Amounts of such powders are not critical, but if used, typically will be present in an amount of about 0.5 to about 5% (w/w).

The sunscreen compositions may also include additional ingredients such as, but not limited to, structuring agents, silicas and silicates, surfactants, pigments, conditioning agents, and dyes.

The sunscreen compositions are applied on a substrate, (i.e., human skin) and is particularly useful as skin-protecting or skin-treatment products for protecting the human skin against the harmful effect of UV radiation. Such sunscreen compositions can be applied to eyes, cheek, chin, neck and other facial areas, and it can also be applied to other bodily areas that are potentially susceptible to sun exposure and UV radiation. The compositions may be in form of a spray, milky lotion, lotion, serum, or light cream.

The method of using the sunscreen compositions will depend on the ultimate intended use of composition. For example, the sunscreen compositions described herein may be applied to the skin on an as-needed basis to provide occasional protection against particularly intense sun light exposures. The sunscreen composition may also be applied to the skin in a regular manner. For example, the sunscreen composition can be applied regularly at a frequency ranging from about once per week to about four or three or two times per day, every two hours or as many times as required or desired. Further, the sunscreen compositions should be applied after swimming or immersion in water and every two hours when exposed to sun.

The following examples describe the compositions and the methods of use of the water-resistant sunscreen composition.

### EXAMPLES

The following compositions provide examples of several embodiments of continuous spray oil-in-water emulsions described herein that provide very water-resistant results.

All compositions below are made according to the following procedure:

1. Phase A and Phase C are separately heated to 75° C.

2. Phase B is premixed and added to Phase A.

3. Phase D is added to Phase C and then combined Phases C and D are then added to combined Phases A and B, and emulsified under homogenization (hereinafter, referred to as Main Phase).

4. Begin cooling the Main Phase to 30° C.

5. As the Main Phase cools, E-I are added to the Main Phase one at a time at suitable temperatures during the cool-down. Suitable temperatures are temperatures known to one skilled in the art, at which the integrity of the ingredients would be maintained.

The process can be modified to adjust the homogenizer speed having high shear rate to reduce the particle size of the emulsion, resulting in different forms of the composition including, milky lotion, watery lotion, lotion, serum, or light cream.

TABLE 1
Exemplary Sunscreen Compositions

| | FOR- | FOR- | FOR- |
|---|---|---|---|
| | MULA | MULA | MULA |
| | 1 | 2 | 3 |
| CTFA NAME | (wt. %) | (wt. %) | (wt. %) |
| Phase A | | | |
| Water | 49.43 | 49.925 | 50.50 |
| Trehalose | 1.0 | 1.0 | 1.0 |
| Sodium Dehydroacetate | 0.1 | 0.1 | 0.1 |
| Tetrasodium EDTA/Disodium EDTA | 0.1 | 0.1 | 0.1 |
| Caffeine | 0.2 | 0.2 | 0.2 |
| Aciylates Copolymer | 0.375 | 0.375 | 0.18 |
| Niacinamide | 1.0 | 1.0 | 1.0 |
| Glycerin | 1.0 | 1.0 | 1.0 |
| Phase B | | | |
| Butylene Glycol | 2.0 | 2.0 | 2.0 |
| Dehydroxanthan Gum | 0.075 | 0.075 | 0.2 |
| Phase C | | | |
| Butyloctyl Salicylate | 4.0 | 4.0 | 4.0 |
| Avobenzone | 3.0 | 3.0 | 3.0 |
| Neopentyl Glycol Diheptanoate | 2.0 | 2.0 | 2.0 |
| VP/Eicosene Copolymer | 0.5 | | 0.5 |
| Tricontanyl PVP | | 0.5 | |
| BHT | 0.09 | 0.09 | 0.09 |
| Dipentaelythrityl Tri-Polyhydroxystearate | 1.25 | 1.25 | 1.25 |
| Homosalate | 7.0 | 7.0 | 7.0 |
| Ethylhexyl Salicylate | 4.5 | 4.5 | 4.5 |
| Tocopheryl Acetate | 0.2 | 0.2 | 0.2 |
| C12-15 Alkyl Benzoate/ | | | |
| Dipropylene Glycol Dibenzoate/ | | | |
| PPG-15 Stearyl Ether Benzoate | 5.0 | 5.0 | 5.0 |
| Polyester-8 | 3.0 | 3.0 | 3.0 |
| Ethylhexyl Methoxycrylene | 2.0 | 2.0 | 2.0 |
| 7-Dehydrocholesterol | 0.01 | 0.01 | 0.01 |
| Methyl Dihydroabietate | 0.5 | | |
| Phase D | | | |
| Potassium Cetyl Phosphate | 2.0 | 2.0 | 2.0 |
| Phase E | | | |
| Phenoxyethanol/Capry1y1 Glycol/ | 1.3 | 1.3 | 1.3 |
| Chlorphenesin | | | |
| Ethylhexylglycerin | 0.3 | 0.3 | 0.3 |
| Phase F | | | |
| Methyl Trimethicone | 4.0 | 4.0 | 4.0 |
| Lamyl PEG-9 Polydimethylsiloxyethyl | 2.5 | 2.5 | 2.5 |
| Dimethicone | | | |
| Cetyl PEG/PPG-10/1 Dimethicone | 0.5 | 0.5 | 0.5 |
| Phase G | | | |
| Algae Extract | 0.05 | 0.05 | 0.05 |
| Phase H | | | |
| Sorbitol | 0.5 | 0.5 | 0.5 |
| Phase I | | | |
| Water | 0.5 | 0.5 | 0.5 |
| Sodium Hyaluronate | 0.02 | 0.02 | 0.02 |

### Test Methods

### In-Vivo SPF Testing Method

All in-vivo SPF measurements were made using the International SPF Test Method (May 2006) COLIPA test methods. SPF static and 80-minute very water-resistant assays were performed on the test subjects. The COLIPA test method is standardized as follows: the irradiation spectrum and the starting output of the sun simulator provided for the test are defined exactly. Additionally, the application amount and the nature of product application are precisely prescribed in the guidelines. The test method is independent of the skin type and the age of the test subjects and can be carried out in a statistical manner at n value greater than or equal to 10 subjects.

The test procedure includes determination of the minimum erythema dose (MED), i.e., the amount of UVB radiation which induces a just perceptible reddening (erythema) for each subject. Then skin on the back of each subject, which include test sites corresponding to untreated, unprotected skin, and test sites corresponding to areas having sunscreen test material, were exposed to radiation. Radiation sources include solar simulators, such as the Modified Solar Ultraviolet Simulator, Model 10S. The pre-immersion and post immersion SPF values were determined according to the COLIPA guidelines.

According to the COLIPA guidelines, a product will be considered very water resistant if the value for the 90% lower unilateral confidence limit is greater than or equal to 50% and the 95% confidence interval on the mean static SPF was within ±17% of the mean static SPF.

### In-Vitro SPF Testing Method

In-vitro testing were performed by preparing the sunscreen test product on polymethylmethacrylate (PMMA) plates (e.g., the Helioplate HD6 available from Lapsphere) and measuring the SPF value using a spectrophotometer (e.g., the SPF-290 Analyzer from Optometrics Corporation). Product application per PMMA plate was approximately 1.3 mg/cm². Pre-immersion SPF value was determined using the spectrophotometer. The sunscreen composition-treated plates were then transferred to a circulating temperature-controlled water bath at 37° C. for 80 minutes. The plates were allowed to dry, and the post-immersion SPF value was then determined.

TABLE 2
Water Resistant Test Results of Formulations in Table 1 (In-Vivo)

| | | POST-IMMER- | WATER |
|---|---|---|---|
| FOR- | PRE-IMMER- | SION | RESISTANT |
| MULA | SION SPF | SPF | (W/R) |
| 1 | 51 | 45 | 88.23% |
| 2 | 52 | 45 | 86.53% |
| 3 | 62 | 50 | 80.64% |

TABLE 3
Water Resistant Test Results of Formulations in Table 1 (In-Vitro)

| | AVERAGE | AVERAGE | WATER |
|---|---|---|---|
| FOR- | ESTIMATED | UVA/UVB | RESISTANT |
| MULA | SPF | RATIO | (W/R) |
| 1 | 51 | 0.8 | 88.23% |
| 2 | 52 | 0.81 | 86.53% |
| 3 | 62 | 0.85 | 81% |

As shown in Tables 2 and 3 above, the sunscreen compositions of Formulas 1, 2, and 3 all satisfy the COLIPA very water-resistant criteria of having a post-immersion SPF value greater than or equal to 50% of the pre-immersion SPF value. Furthermore, each of the in-vivo tests shown above meets the confidence level criteria of ±17%.

Compositions that are shown and tested below in Tables 4 and 5 are similar to Formula 1 shown in Table 1 above, with variations in the film formers used.

TABLE 4
Water Resistant Test Results for Compositions with Varying Ratios of
Acrylates Copolymer to Xanthan Gum (In-Vitro)

| ACRYLATES | | | |
|---|---|---|---|
| COPOLYMER: | AVERAGE | AVERAGE | WATER |
| XANTHAN | ESTIMATED | UVA/UVB | RESISTANT |
| GUM | SPF | RATIO | (W/R) |
| 15:1 | 45 | 0.82 | 100% |
| 7:1 | 50 | 0.79 | 100% |
| 5:1 | 51 | 0.80 | 88% |
| (formula 1 above, | | | |
| as control) | | | |
| 3:1 | 51 | 0.79 | 93% |
| 2:1 | 55 | 0.81 | 79% |
| 1:1.1 | 62 | 0.85 | 81% |
| (formula 3 above) | | | |

TABLE 5
Water Resistant Test Results for Compositions with Varying Ratios of
PVP Polymer to Rosinate Resin (In-Vitro)

| PVP | | | |
|---|---|---|---|
| POLYMER: | AVERAGE | AVERAGE | WATER |
| RONSINATE | ESTIMATED | UVA/UVB | RESISTANT |
| RESIN | SPF | RATIO | (W/R) |
| 15:1 | 56 | 0.83 | 74% |
| 5:1 | 57 | 0.83 | 78% |
| 1:l | 51 | 0.84 | 88% |
| (formula 1 above, | | | |
| as control) | | | |
| 1:5 | 51 | 0.80 | 84% |
| 1:15 | 51 | 0.83 | 77% |

As shown above in Table 4 above, compositions having a ratio of the acrylates copolymer to the dehydroyxanthan gum in the range of 15:1 to 1:1.1 provide varying levels of water resistance that meet the COLIPA very water resistant criteria. Furthermore, as shown in Table 5 above, compositions having a ratio of PVP polymer to the rosinate resin in the range of 15:1 to 1:15 also provide varying levels of water resistance that meet the COLIPA very water-resistant criteria. Compositions shown and tested below are similar to formula 3 in Table 1 above, with variations in the amount of tricontanyl PVP used.

TABLE 6
Water Resistant Test Results for Compositions with Varying
Amount of Tricontanyl
PVP (in-vitro) by the total weight of the composition

| | AVERAGE | AVERAGE | WATER |
|---|---|---|---|
| TRICONTANYL | ESTIMATED | UVA/UVB | RESISTANT |
| ARIATIONS | SPF | RATIO | (W/R) |
| 0.075% | 51 | 0.79 | 97% |
| 0.20% | 47 | 0.81 | 99% |
| 0.5% | 52 | 0.81 | 87% |
| (formula 2 above, | | | |
| as control) | | | |
| 1.0% | 52 | 0.80 | 92% |
| 2.0% | 58 | 0.81 | 79% |

As seen from Table 6 above, compositions having a range of about 0.075% to about 2.0% (w/w) of tricontanyl PVP by the total weight of the composition provide varying levels of water resistance that meet the COLIPA very water-resistant criteria.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the scope of the invention. It is therefore intended to cover in the appended claims all such changes and modifications that are within the scope of this invention.

What is claimed is:

1. A sunscreen composition, comprising:
one or more sunscreen actives; and
at least two film formers comprising an acrylates copolymer and a xanthan gum in a ratio from about 15:1 to about 1:1, wherein
the sunscreen composition provides very water-resistant properties upon application on a substrate and subsequent to water immersion of the substrate.

2. The sunscreen composition of claim 1, wherein the sunscreen composition is free of oxybenzone and octocrylene.

3. The sunscreen composition of claim 1, wherein the xanthan gum comprises dehydroxanthan gum.

4. The sunscreen composition of claim 1, wherein the sunscreen composition further comprises a polyvinylpyrrolidone (PVP) polymer, wherein the polyvinylpyrrolidone (PVP) polymer is VP/eicosene copolymer or tricontanyl PVP.

5. The sunscreen composition of claim 4, wherein the tricontanyl PVP is present in an amount from about 0.075% to about 2.0% (w/w) by the total weight of the sunscreen composition.

6. The sunscreen composition of claim 4, wherein the sunscreen composition further comprises a rosinate resin, wherein the rosinate resin is methyl dihydroabietate.

7. The sunscreen composition of claim 6, wherein the polyvinylpyrrolidone (PVP) polymer and the rosinate resin is present in a ratio of about 15:1 to about 1:15.

8. The sunscreen composition of claim 1, wherein the one or more sunscreen actives comprise at least one organic sunscreen active.

9. The sunscreen composition of claim 8, wherein the at least one organic sunscreen active is selected from the group consisting of avobenzone, homosalate, salicylate derivatives, and combinations thereof

10. The sunscreen composition of claim 1, wherein one or more sunscreen actives are present in an amount of about 3% to about 30% (w/w) by the total weight of the sunscreen composition.

11. The sunscreen composition of claim 1, wherein an SPF rating from about 75% to about 100% is retained for at least eighty minutes upon application of the sunscreen composition and after immersion in water.

12. A method of protecting skin from ultraviolet radiation, comprising:
applying a sunscreen composition to the skin, the sunscreen composition comprising:
one or more sunscreen actives; and
at least two film formers comprising an acrylates copolymer and a xanthan gum in a ratio of about 15:1 to about 1:1.

13. The method of claim 12, wherein the sunscreen composition further comprises a film former selected from the group consisting of a polyvinylpyrrolidone (PVP) polymer, a rosinate resin, and combinations thereof

14. The method of claim 13, wherein the ratio of the polyvinylpyrrolidone (PVP) polymer to the rosinate resin is from about 15:1 to about 1:15.

15. The method of claim 13, wherein the polyvinylpyrrolidone (PVP) polymer is VP/eicosene copolymer or tricontanyl PVP.

16. The method of claim 15, wherein the tricontanyl PVP is present in the sunscreen composition in an amount of about 0.075% to about 2.0% (w/w) by the total weight of the sunscreen composition.

17. The method of claim 12, wherein the sunscreen composition is free of oxybenzone and octocrylene.

18. The sunscreen composition of claim 1, wherein the sunscreen composition is a spray, lotion, light cream or continuous lotion spray.

19. The method of claim 12, wherein the sunscreen composition is a spray, lotion, light cream or continuous lotion spray.

20. The method of claim 12, wherein an SPF rating from about 75% to about 100% is retained for at least eighty minutes upon application of the sunscreen composition on the skin and after immersion in water.
